(19) United States
(12) Patent Application Publication
(10) Pub. No.: US 2020/0209180 A1
(43) Pub. Date: Jul. 2, 2020

(54) SOLID STATE ELECTRODES, METHODS OF MAKING, AND METHODS OF USE IN SENSING

(71) Applicant: Razzberry, Inc., New Hacen, CT (US)

(72) Inventors: Alexandra Barton-Sweeney, New Haven, CT (US); Honest Makamba, New Haven, CT (US)

(21) Appl. No.: 15/752,334

(22) PCT Filed: Aug. 12, 2016
(86) PCT No.: PCT/US2016/046714

§ 371 Date: Feb. 13, 2018

(51) Int. Cl. G01N 27/30; G01N 33/94

(52) U.S. Cl. CPC G01N 27/301; G01N 33/94

(57) ABSTRACT

A solid state electrode includes a metal electrode having a surface; a nanocomposite coated on at least a portion of the surface, the nanocomposite comprising a compound of the metal used in the electrode, and nanoparticles, a protein, a polymer, or one or more of nanoparticles, a protein, and polymer; wherein when the solid state electrode is in electrical connection with a working electrode and a fluid, the electrode can detect a change in chemical composition, for example, a change in pH of less than or equal to 0.1 pH units, and the potential of the solid state electrode is stable to within 5 millivolts, such as within 3 millivolts over a period of 20 minutes. The solid state electrode can be used in biosensing, environmental analysis (e.g., soil analysis, or water analysis), pharmaceutical analysis, and food analysis, for example.

### CROSS-REFERENCE TO RELATED APPLICATIONS

This application claims priority to U.S. provisional patent application 62/205,380, filed Aug. 14, 2015; U.S. provisional patent application 62/254,402, filed Nov. 12, 2015; U.S. provisional patent application 62/290,501, filed Feb. 3, 2016; and U.S. provisional patent application 62/322,273, filed Apr. 14, 2016, the contents of each of which are hereby incorporated by reference in their entirety.

### BACKGROUND

Electrodes are used in electrochemical sensing applications. The technology of conventional, macro-sized electrodes has been developed over a long period of time. With the advance of miniaturized technologies for portable, micro-sized electrochemical sensors, the electrodes themselves have to be miniaturized as well.

For example, conventional macro-sized reference electrodes in aqueous/wet conditions typically use a silver/silver chloride composite with a potassium chloride solution that helps to stabilize the silver chloride that is coated on the silver wire. Additionally, they must be wet at all times. This arrangement cannot be used for micro-sized reference electrodes (microelectrodes) due to the small space available in a micro-sized electrochemical sensor, which complicates miniaturization of a separate solution system of potassium chloride that would stabilize the solid state. Therefore a solid state electrode is the preferred type of micro-sized electrode. For desired performance, the potential of a reference electrode should be stable or invariant during electrochemical sensing. One problem with current reference solid state electrodes is that their potential is not stable. The instability is due to the fact that the silver chloride is dissolved during operation.

Conventional macro-sized working electrodes, especially those requiring the use of a membrane such as an ion selective electrode, also pose challenges when miniaturizing, because conventional working electrode membranes also require a separate solution system. Additionally, chemically selective membranes, such as ion selective electrode membranes, deposited on solid state working electrodes are known to present problems of instability. This results in shifts in the measured potential of the working electrode. The instability of the potential is thought to be caused by patches that are formed at the membrane/working electrode interface. The patches cause random collection of water at the interface, which results in the variation of the amount of the analyte that can reach the interface. Solutions that have been reported for this problem include the use of conducting polymers as interlayer films between the electrode and the ion selective membrane. However, these interlayer films create other issues, such as environmental sensitivity, including sensitivity to light or sample changes, such as pH shifts, which create problems when detecting chemical concentrations in samples that have changing compositions, such as in the environment or in biofluids, such as bodily fluids.

Therefore, there remains a need in the art for stable solid state electrodes, including stable solid state reference and working electrodes, for applications such as in aqueous conditions. Stable solid state electrodes are important for the field of non-invasive health diagnostics using sweat sensing, for example, where recent reports have shown that the concentration of various biomarkers in sweat correlates with their concentration in blood. This means that a wearable sweat sensor could provide a non-invasive way to continuously track the health of humans with serious diseases.

### SUMMARY

A solid state electrode comprising: a metal electrode having a surface; a nanocomposite coated on at least a portion of the surface, the nanocomposite comprising:

a compound of the metal used in the electrode, and nanoparticles, a protein, a polymer, or a combination comprising at least one of the foregoing; wherein when the solid state electrode is in electrical connection with a working electrode and a conductive substance, the solid state electrode can detect a change in chemical composition of the conductive substance, and the potential of the solid state electrode is stable. In an embodiment, the potential of the solid state electrode is stable to within 5 millivolts, preferably within 3 millivolts over a period of 20 minutes. As an example, a potential change of 1 mV, or 0.5 mV, or 0.1 mV per minute over a period of time, is stable. In an embodiment, a potential change of 0.1 mV per minute or less over 20 minutes is stable. In an embodiment, a potential change of 0.1 mV per minute or less over 30 minutes is stable. In an embodiment, the system uses amperometric sensing with biorecognition element, as described further herein.

A method of making a solid state electrode, comprising: providing a metal electrode having a surface; attaching a nanocomposite comprising a compound of the metal used in the electrode, and nanoparticles, a protein, a polymer, or a combination comprising at least one of the foregoing, onto at least a portion of the electrode surface is provided. A biosensor for determining a parameter of a conductive substance, comprising: a substrate having a top surface and a bottom surface; a working electrode comprising a membrane, a selective membrane, an ion-selective membrane or a metal oxide, disposed on the top surface of the substrate; a reference electrode comprising the solid state electrode described here, disposed on the surface of the substrate, wherein the working electrode and reference electrode are electrically coupled when in contact with a conductive substance is provided.

Disclosed herein is a reference solid state electrode comprising: a metal electrode having a surface; a nanocomposite coated on at least a portion of the surface, the nanocomposite comprising (a) a compound of the metal used in the metal electrode, and (b) nanoparticles, a protein, a polymer, or a combination comprising at least one of nanoparticles, a protein, and a polymer; wherein when the solid state electrode is in electrical connection with a working electrode and a conductive substance, such as a fluid, metal, or gel, the solid state electrode can detect a change in chemical composition or detect a chemical composition level of the conductive substance, and the potential of the solid state electrode is stable.

The conductive substance can be any solution, such as a fluid, which can be any biofluid such as a bodily fluid (e.g., sweat, blood, saliva, urine, sebum, or other excretions). The conductive substance can also be a conductive material, metal, or gel.

The change in chemical composition can be, for example a change in hydrogen ions (pH) or the determination of a chemical level, for example the level of hydrogen ions (pH). Any chemical changes can be detected using this reference solid state electrode where the sensor is modified to select for the specific chemical, such as ions (e.g. chloride, magnesium, potassium, sodium, hydrogen, calcium, ammonium, carbonate, nitrates), enzymes, proteins, lipids, bicarbonate levels, chemical compounds (e.g. DNA, RNA, creatinine, urea, glucose), acids, foreign substances (e.g. toxins, such as arsenic, cyanide, amphetamines, drugs, ethanol), xenometabolites, and any other analyte that can be analyzed electrochemically or in a fluid, such as those described further herein.

Electrodes described herein comprise a metal. The metal in the electrode can be gold, mercury, platinum, silver, palladium, copper, or a combination comprising at least one of the foregoing. A compound of a metal used in the reference electrode or other electrode can be an ionic or covalently bonded compound comprising the metal. In an embodiment, a compound of a metal used in the reference electrode is a salt, such as a chloride salt, an iodide salt, a sulfate salt, or other salt of the metal used in the reference electrode. In embodiments, a compound of a metal used in the reference electrode can be mercury chloride, silver chloride, silver iodide, copper sulfate, mercurous sulfate, or a combination comprising at least one of the foregoing. As an example, if the electrode comprises gold, a compound of a metal used in the electrode comprises gold, and the compound of a metal used in the electrode can be a salt of gold, such as sodium aurothiosulfate. The proteins can be any protein that exhibits strong binding characteristics, such as adhesive proteins, mussel proteins, fibrinogen, protofilaments, amyloid nanofibrils, or a combination comprising at least one of the foregoing. The polymers can include PVB (polyvinyl butyral) or any polymer that exhibits strong binding characteristics. The nanoparticles can be gold nanoparticles, silver nanoparticles, copper nanoparticles, zinc oxide nanoparticles, carbon nanoparticles, spherical carbon nanoparticles, fullerenes, quantum dots, graphene oxide, carbon nanotubes, nano clusters, nanofibers, carbon nanofibers, diamond nanoparticles, carbon quantum dots, titanium oxide nanoparticles, titanium dioxide (TiO₂) nanoparticles, silicon oxide nanoparticles, gold nanoclusters, silver nanoclusters, europium oxide nanoparticles, iron oxide nanoparticles, diamond nanoparticles, inorganic quantum dots, graphene quantum dots, graphene nanoparticles, or a combination comprising at least one of the foregoing. Unless otherwise indicated, “strongly binding” or other forms of the phrase means binds sufficiently to allow the desired interactions to occur, or for the desired functions to occur, as described herein.

In an embodiment, the reference solid state electrode comprises a metal electrode, a compound of the metal used in the metal electrode, and also includes at least one of nanoparticles, a polymer, and a protein, or a combination comprising at least one of nanoparticles, a polymer, and a protein. One or more of each of nanoparticles, polymers, and proteins can be used, such as one or more nanoparticles, one or more proteins, or one or more polymers. In an embodiment, the reference solid state electrode comprises a compound of the metal used in the metal electrode, and also comprises one or more nanoparticles. In an embodiment, the reference solid state electrode comprises a compound of the metal used in the metal electrode, and also comprises one or more polymers. In an embodiment, the reference solid state electrode comprises a compound of the metal used in the metal electrode, and also comprises one or more proteins.

As used herein, “solid state electrode” means an electrode that does not contain liquid solutions or liquids in its structure.

While the description herein primarily refers to building and operating a reference solid state electrode, it should be understood that the approaches described here are not limited. Any of the approaches described can be applied to building and operating any of the other solid state electrodes in a sensor as well, including a working electrode, a counter electrode, and others.

Also disclosed herein is a method of making a reference solid state electrode, comprising: providing a metal electrode having a surface; attaching a nanocomposite comprising a compound of the metal used in the metal electrode; and nanoparticles, a protein, polymer, or a combination comprising at least one of nanoparticles, PVB, and an adhesive protein, onto at least a portion of the metal electrode surface. The nanocomposite can comprise a compound of the metal used in the metal electrode; and nanoparticles, one or more proteins, a polymer, or a combination comprising at least one of nanoparticles, a polymer, and a protein.

Also disclosed herein is a biosensor for determining a parameter of a conductive substance, such as a fluid, comprising: a substrate having a top surface and a bottom surface; a working electrode comprising an ion-selective membrane or a metal oxide disposed on the top surface of the substrate, or another chemically-selective membrane modified to bind with the specific chemical or analyte; a reference electrode comprising a metal electrode having a surface; a nanocomposite coated on at least a portion of the surface, the nanocomposite comprising a compound of the metal used in the metal electrode, and nanoparticles, one or more proteins, a polymer, or a combination comprising at least one of nanoparticles, one or more proteins, and a polymer; wherein when the solid state electrode is in electrical connection with a working electrode and a conductive substance, such as a fluid, the electrode can detect a change in an analyte or a concentration level of an analyte, for example, a change in pH of less than or equal to 0.5 pH units, in one embodiment less than or equal to 0.05 pH units, and the potential of the electrode is stable with a potential change over time of 0.1 mV per minute or less, over a time of 20 minutes or more, disposed on the top surface of the substrate, wherein the working electrode and reference electrode are electrically coupled when in contact with a conductive substance, and the biosensor can be attached to the skin of a human or mammal, for example, through the bottom or top surface of the substrate. The solid state reference electrode will remain stable during changes in concentration of the fluid, even when selecting for an analyte under changing conditions such as fluctuations in temperature, pH, and changes of other analytes in the fluid. The conductive substance can be a fluid such as a bodily fluid, or other fluid, such as a laboratory or environmental water sample, for example; a gel; a metal; a material, or any other conductive substance.

The above described and other features are exemplified by the following figures and detailed description.

### BRIEF DESCRIPTION OF THE FIGURES

The following Figures are exemplary embodiments.

FIG. 1 shows the pH stability of five devices (D1-D5) prepared using the methods described herein at four different pH values.

FIG. 2 shows the pH stability of two graphene oxide devices (D1 GO, D2 GO) at two different pH levels.

### DETAILED DESCRIPTION

Described generally herein are working and reference solid state electrodes, methods of making the solid state electrodes, and methods of using the solid state electrodes, for example, in a biosensor.

It should be appreciated that the solid state electrodes and biosensor described herein can be used in many applications and devices, such as a sensing device or system for measuring ions, biomarkers, enzymes, compounds, DNA, RNA, proteins, drugs, toxins, metabolites, or other chemical species in human or mammalian sweat. The solid state electrodes and biosensor may be used in the device described in commonly owned U.S. Nonprovisional application Ser. No. 14/662,411, filed Mar. 19, 2015, entitled “Health State Monitoring Device,” the contents of which are incorporated herein by reference in its entirety. Besides biosensing, the solid state electrodes can be used in other areas where microfluidic chips are used, such as environmental analysis (e.g., soil analysis, or water analysis), explosives analysis, pharmaceutical analysis, and food analysis.

The solid state electrodes can be of any suitable size and shape. For example, the electrode can be a wire, a thin film on a surface, a pattern on a flexible substrate, a material, or ink. The electrode may be part of a printed sensor. The electrode can be any suitable thickness that allows the desired formation steps to occur and also allows fabrication into a desired device. The nanocomposite can be coated on substantially all or a portion of the surface of the electrode. For example, the electrode can be a generally two-dimensional shape, and the nanocomposite can be coated on one side, or a portion of one side of the metal electrode. Coating does not necessarily mean a uniform layer is formed. There may be holes, voids, or other areas where there is no nanocomposite or less nanocomposite or more nanocomposite than in other areas, as long as the nanocomposite coated surface performs in the desired manner and with the desired characteristics, as described herein.

The nanocomposite can comprise a compound of a metal, such as the metal used in the electrode, and also either nanoparticles, a protein or proteins, a polymer or polymers, or a combination comprising at least one of nanoparticles, a protein, and a polymer.

The carbon nanoparticles can be made from different sources. They can be made from sources such as amino acids, non-amino organic acids, alcohols, alkanes, monosaccharides, and biological materials. Specific sources include methane, ethanol, ethane, citric acid, gluconic acid, glucuronic acid, glucosamine, galactosamine, fructosamine, mannosamine and other carbon sources such as eggs. The carbon nanoparticles can be of any suitable form, for example, carbon nanotubes (single-wall or multi-wall), graphene, fullerenes, diamond, carbon quantum dots, graphene quantum dots, amorphous carbon, or carbon nanofibers, or a combination comprising at least one of the foregoing. The carbon nanoparticles can be graphitic in structure, such as flat, disk-shaped, cylindrical, spherical, or irregularly shaped. Carbon nanoparticles can be fluorescent or non-fluorescent.

The carbon nanoparticles can be modified, for example, where a hydrophobic compound comprising an amine group and a thiol group is covalently bonded to the carbon nanoparticles. Further, the carbon nanoparticles can be modified with a hydrophobic compound containing a carboxylic group and a thiol. This modification can be carried out using conventional methods, such as carbodiimide coupling or Schiff base conjugation. The hydrophobic compound comprising an amine group and a thiol group can be any one of a number of compounds, such as 4-aminothiophenol or 5-amino-2-mercaptobenzimidazole. The hydrophobic compound comprising a carboxyl group and a thiol group can be 5-carboxy-2-mercaptobenzimidazole or compounds with a similar structure. The hydrophobic compound comprising an amine group and a thiol group can also include an aromatic group, which can reduce the solubility of the compound of the metal used in the electrode.

Hydrophobic compounds can include amine, thiol, aromatic, and carboxyl groups, such as 4-aminothiophenol, 5-amino-2-mercaptobenzimidazole, 5-carboxy-2-mercaptobenzimidazole, thiophenol, 2-Napthalenethiol, and 9-Anthracenethiol.

The nanoparticles can have any suitable size and shape as long as the nanoparticles function in the desired methods and do not interfere with the operation of the solid state electrode. The nanoparticles are generally small, such that they may have an average diameter of less than or equal to 100 nanometers, in one embodiment less than or equal to 50 nanometers, in another embodiment less than or equal to 20 nanometers, in another embodiment less than or equal to 15 nanometers, and in still another embodiment less than or equal to 10 nanometers.

The nanocomposite can include a protein, such as an adhesive protein or amyloid nanofibrils, or a mixture of proteins, such as a mixture of adhesive proteins or amyloid nanofibrils. One example of a protein is an adhesive protein such as a mussel protein, which can be considered as a natural glue. The amount of the protein used in the nanocomposite can vary, but can be 0.7 milligram per milliliter (mg/ml) or lower, in one embodiment 0.5 mg/ml or lower, and in still another embodiment 0.1 mg/ml or lower.

The nanocomposite can include proteins such as amyloid type nanofibrils, also known as protofilaments. Proteins and polypeptides such as fibrinogen can assemble to form amyloid type nanofibrils. The amount of amyloid type nanofibril can vary, but can be 0.7 milligram per milliliter (mg/ml) or lower, in one embodiment 0.5 mg/ml or lower, and in still another embodiment 0.1 mg/ml or lower. If the nanocomposite includes an adhesive protein or mixture of adhesive proteins, and an amyloid type nanofibrils or mixture of amyloid type nanofibrils, the concentration of each component can vary, but in one embodiment, the total concentration of an adhesive protein or mixture of adhesive proteins, and an amyloid type nanofibril or mixture of amyloid type nanofibrils can be 0.7 milligram per milliliter (mg/ml) or lower, in one embodiment 0.5 mg/ml or lower, and in still another embodiment 0.1 mg/ml or lower, and each component in the nanocomposite can have any concentration in the total.

A method of making a reference solid state electrode is provided, comprising: providing a metal electrode having a surface; attaching a nanocomposite comprising a compound of the metal used in the metal electrode, and nanoparticles, one or more proteins, and a polymer, or a combination comprising at least one of nanoparticles, onto at least a portion of the metal surface.

The nanocomposite can be attached to the surface of the metal electrode using either physical deposition or electrochemical deposition.

Physical deposition refers to any method, including chemical deposition that does not use a voltage to attach the nanocomposite to the surface of the electrode. The compound of the metal used in the electrode can be produced by oxidation of the metal surface by using an oxidizing agent. The oxidizing agent can be washed away after the deposition of the layer comprising the compound of the metal used in the electrode, or layer comprising a compound of the metal used in the electrode and nanocomposite. In one example, physical deposition comprises mixing the nanoparticles with the oxidizing agent in a solution, to form a composite solution, and applying the composite solution to the surface to produce a composite solid state electrode. The concentration of the oxidizing agent can be any suitable concentration to achieve the desired results, and can be 0.5 Molar (M)±0.25 M, and in one embodiment 0.1 M±0.05 M.

The oxidizing agent can be permanganate, dichromate, iron (III) chloride, perchlorate, periodate, hydrogen peroxide, chlorate, chromate, or iodate.

The nanocomposite can include a protein, one or more polymers, or nanoparticles, or a combination comprising at least one of the foregoing, and the protein, one or more polymers, or nanoparticles, or combination, can be mixed with the oxidizing agent prior to attaching the nanocomposite onto the surface.

Electrochemical deposition uses a voltage to attach the nanocomposite to the surface of the electrode. Electrochemical deposition can include applying a voltage or current to the surface in an acid solution, forming a coated surface of the compound of the metal used in the metal electrode; and electrochemically depositing the nanocomposite onto the compound of the metal used in the metal electrode coated surface. In one example, electrochemically depositing includes applying a voltage or current to the surface, forming a compound of the metal used in the metal electrode nanoparticle composite coated surface. As an example, the electrochemical deposition can done by applying 20 μA for 1 minute or 2 minutes. The acid solution can be sulphuric acid solution, nitric acid solution, potassium chloride, acidified potassium chloride, potassium chloride acidified with hydrochloric acid, hydrochloric acid solution, phosphorous, or phosphoric acid.

A biosensor for determining a parameter of a fluid, such as a bodily fluid, can include a substrate having a top surface and a bottom surface; a working electrode comprising an ion-selective membrane or a metal oxide, or another chemically-selective membrane modified to bind with the specific chemical or analyte, disposed on the top surface of the substrate; a reference electrode comprising an electrode as described herein, disposed on the top surface of the substrate, wherein the working electrode and reference electrode are electrically coupled when in contact with a bodily fluid or other fluid to be measured, and wherein the biosensor can be attached to the skin of a human or mammal through the bottom or top surface of the substrate, for example, or wherein the working electrode can be attached to or contacted with the skin of a human or mammal.

One of the surfaces can be electrically connected to a device. The connection can be via any form of electrical connection, via soldering, pads, pins, etc.

The bodily fluid can be sweat, saliva, blood, urine, sebum, tears, skin interstitial fluid, or any other secretions, including vaginal fluids, semen, menstrual blood, cerebrospinal fluid, lymph, breast milk, cerumen/ear wax, feces, vomit, bile, or mucus. The parameter of a bodily fluid can be the level of H⁺(pH), Na⁺, Mg²⁺, NO₃⁻, NH₄⁺, K⁺, Ca²⁺, Cl⁻, CO₃²⁻, testosterone, follicle stimulating hormone (FSH), estrogen, urea, creatinine, progesterone, androstenedione, glucose, cytokines, DNA, RNA, proteins or beta-human chorionic gonadotrophin (hCG), compounds, for example. The parameter of a bodily fluid can also be cortisol, creatinine, urea, glucose, lactic acid, acids, salts, cations, cytokines, dopa, dopamine, drugs, opiates, buprenorphine, amphetamines, gamma hydroxybutyrates, ethanol, cocaine, alcohols, metabolites, xenometabolites, dioxins, xenobiotics, organic compounds, mycotoxins, metals, zinc, lead, mercury, cadmium, pthalates, arsenic, cyanide, BPA, environmental toxins, industrial metals and toxins. While these analytes are listed for sensing bodily fluids, they should not be so limited and can include sensors for detecting analyte levels in a variety of samples, such as water for environmental analysis, or food samples. The biosensor can measure a parameter such as a biomarker that may be correlated with a fertility state of a human or mammal, in particular a human or mammalian female. The bio sensor can measure changes of ions that indicate a disease state or a nutritional deficiency, for example. The biosensor can be used to provide measurements similar to a blood panel, without an invasive test. The biosensor can measure changes in biomarkers that are correlated with a disease state, for example glucose for diabetes, or chloride for cystic fibrosis. Chloride can be measured using a membrane selective electrode while other analytes can be measured by modifying the working electrode with a specific bio-recognition element. Examples of bio-recognition elements are glucose oxidase for glucose sensing by a redox process, urease for urea sensing, creatinine deiminase for creatinine sensing and calmodulin for calcium sensing, in which calmodulin undergoes a conformational change. Urea and creatinine can be sensed indirectly by measuring the concentration of ammonium ions released during the enzymatic processes. Cytokines, which are biochemicals that indicate the state of cells can also be measured with the biosensor. Cytokines are known to be biomarkers for cancer, infection, and trauma among others. Cytokines are released in very low concentrations in bodily fluids. For example, in an embodiment, a solid state reference electrode comprises: a metal electrode having a surface; a nanocomposite coated on at least a portion of the surface, the nanocomposite comprising a compound of the metal used in the metal electrode, and nanoparticles, a protein, a polymer, or a combination comprising at least one of the foregoing, and a solid state working electrode wherein the electrode is modified with a bio-recognition element, or a redox couple, such as the use of glucose oxidase for glucose sensing by a redox process, to detect a specific analyte; wherein when the solid state reference electrode is in electrical connection with the working electrode and a conductive substance, the electrode can detect a change in an analyte), and the potential of the electrode is stable to within 5 millivolts, such as within 3 millivolts over a period of 20 minutes.

Some metabolites produced in the body are thought to affect genes. The biosensor can be used to analyze these metabolites, which are small molecules that may be detected electrochemically. Sweat composition can indicate the state of vital body systems, such as the muscles, heart, kidney, digestion, lungs, thyroid, and brain. Studies have shown that sweat composition variations and the existence of certain biomarkers can indicate the existence, onset of, or tendency toward certain health conditions. High levels of sweat potassium are early markers for heart disease and kidney failure. Too little calcium can indicate a vitamin D deficiency or thyroid disorder. Elevated sweat calcium levels are commonly associated with cancer, e.g. lung cancer. Ion fluctuations indicating cyclic patterns during the menstrual cycle and fertile window, as well as the onset of menopause, pre-eclampsia during pregnancy; impact of antibiotics, chemotherapy, and radiation treatments; concussion, stroke, intestinal distress, malnutrition, vitamin and mineral deficiencies, stress, both physical and psychological, and many other health conditions or physiological states. The existence of certain DNA and RNA and other analytes in sweat can indicate the presence of certain cancers, and whether they have metastasized, as well as allergens, and other health conditions. The biosensor can also be used to monitor or diagnose Parkinson's disease, or dopa or dopamine levels. The biosensor can also include additional sensors of any kind, for example sensors for measuring humidity, heart rate, motion, impedance, and temperature, for example.

Many other substances are also present in sweat: nitrogenous compounds such as amino acids and urea, metal and nonmetal ions such as potassium, sodium, and chloride ions; metabolites including lactate and pyruvate; compounds, such as glucose, and xenobiotics such as drug molecules or poisons, such as arsenic or cyanide, pollution, chemical and environmental toxins, including mycotoxins, organic compounds, BPA, pthalates, heavy metals such as arsenic, cadmium, lead and mercury. In a diseased or unnatural state, sweat may contain additional analytes or disease-linked biomolecules, such as those specific to a particular condition or exposure.

The sensors can be used to detect analytes as part of a Metabolic Panel (or any combination of other metabolites or analytes, such as glucose, etc). This could be used as a replacement or in conjunction with a blood or urine Basic Metabolic Panel or Creatinine/Albumin or Creatinine/Blood Urea Nitrogen test.

A Basic Metabolic Panel generally includes any combination or subset of the following analytes: Chloride, Potassium, Sodium, Bicarbonate, Creatinine, and BUN (Blood Urea Nitrogen). A microelectrode in the form of a patch to monitor the basic metabolic panel of sweat can use a combination of sensors to detect Chloride, Potassium, Sodium, Creatinine, Carbonate, and Urea. Urea can be indirectly detected by measuring ammonium, a by-product of urea breakdown by the enzyme urease.

Urea can be analyzed electrochemically using the enzyme urease on the electrode or in combination with nanomaterials. Urea can be determined in sweat, and has been found to have sweat levels that are 3.6 times that in serum. In another embodiment, a means of measuring urea concentration in a biofluid, detects byproducts of urea instead of directly detecting urea. For example, urease enzyme breaks down urea to produce bicarbonate and ammonium. The urease enzyme can be attached to an ion selective membrane on a working microelectrode that can detect the ammonium by-product.

Molecularly imprinted polymers can be used for electrochemical analysis of an analyte, for example the use of molecularly imprinted polymers for urea and creatinine detection. In an embodiment, urea can be imprinted in polymer, and the imprinted polymer is then incorporated into a urea selective membrane using standard procedures that are used for making ion selective membranes.

An integrated device can be used in which analytes, such as creatinine are detected using an enzyme, such as creatinine deiminase. In an embodiment, analyte measurement can be enhanced or sensed indirectly by sensing the by-products of the analyte. With creatinine, as an example, ammonium ions are produced, and the ammonium ions can be detected by an electrode with a membrane containing a polymer imprinted with creatinine. Similarly to urea, one can perform analysis of creatinine using a combination of creatinine deaminase with ammonium selective membrane immobilized on the working electrode.

### Dialysis/Kidney Function

Currently, kidney function is determined by a blood draw measuring several biomarkers such as potassium, chloride, carbonate, and sodium. These same biomarkers can be detected in sweat by sensors as described herein and can be used in a low-cost application to allow patients in general, but importantly kidney disease and heart disease patients and those on dialysis or who have had transplants, to track their kidney function on an on-going basis and adjust the amount of dialysis, or medication, or other treatment according to their individual needs, as well as provide early diagnosis of kidney problems, renal failure, the conditions prior to or during a stroke or cardiac events.

### Anti-Doping/Illicit Drugs/Alcohol Abuse

Sweat is an ideal bodily fluid for anti-doping testing. The volume of sweat perspired by the whole human body in one day is 300-700 mL. Many illegal substances, which have been reported to be excreted through sweat in a quantifiable amount, and can be measured with the sensors described here are: opiates, buprenorphine, amphetamines, gamma hydroxybutyrates, ethanol, and cocaine. The detection period for many drugs is only 2-3 days in urine, however, these drugs can be detected for weeks in sweat. In addition, these analytes could be measured in sweat as a function of time. For example, ethanol concentration in sweat measured as a function of time can be used to analyze alcohol ingestion and absorption levels.

### Occupational/Environmental Toxin Exposure

Many toxic metals and substances are excreted via perspiration. Many of these metals are converted to their xenometabolites (cations or salts) in the body followed by their solubilization in sweat. The excreted sweat concentrations of some metals (e.g., cadmium and lead) are sometimes comparable to those of urine; thus sweat can be used as an alternative to urine testing. Because the sensor described herein can detect metal ions, it can therefore be adapted to detect lead, mercury, cadmium, as well as other poisonous substances, such as arsenic, cyanide, and other poisonous substances.

### Drug Accumulation in Sweat

Sweat glands act as excretory organs, like the kidneys and lungs, for drugs and their metabolites. Many drugs have a basic pKa and are known to accumulate in sweat more than in blood, due to the higher acidity of sweat. The concentration of drugs in the sweat can be used to determine dosage information, including over and under medication, as well as overdosing, drug behavior and performance. This has applications in clinical trials, pharmacokinetics, forensics, and drug testing.

The transport of water insoluble drugs between blood and other biofluids depends on the pH of the other biofluids and the drug's pKa which are helpful in theoretical computation of the biofluid-to-plasma concentration ratio of drug using Henderson-Hasselbach equation. The concentration gradient between plasma and sweat provides driving force for passive diffusion of the free fraction of drug from plasma to sweat through lipid bilayer.

### Other Health Applications

Diabetic markers have been shown to exist in sweat, including a correlation between sweat composition and sweat glucose to blood glucose levels. Researchers have observed differences between lung cancer patient sweat metabolites and healthy subjects. Dermcidin (DCD), a peptide containing 47-amino acids, and prolactin inducible protein (PIP), have been shown to be prognostic markers and are present in sweat. DCD has been shown to promote cell growth in tumorigenesis and is over expressed in the presence of invasive breast carcinomas and lymph node metastases. PIP has been shown to be overexpressed in metastatic breast and prostate cancer. Other prognostic biomarkers in sweat have been used in the diagnosis of Schizophrenia, Cystic Fibrosis, and recently have been linked to Parkinson's disease.

The sensors described herein can be used to detect the substances described herein.

The biosensor can be any suitable size and shape. For example, the biosensor can be between 0.5 and 10 millimeters thick.

The working electrode, reference electrode, and other components of the biosensor can be deposited on a substrate by techniques such as screen printing, roll-to-roll printing, aerosol deposition, inkjet printing, thin film deposition, or electroplating. The substrate can be a flexible or rigid polymer, a textile, a mat, glass, metal substrate, or other printed material. The substrate can be non-electrically conductive. The substrate can be electrically conductive. There may be intermediate layers between the substrate and electrodes.

The solid state electrodes, methods, and biosensors are further illustrated by the following non-limiting examples.

Special equipment may be made to build the sensors and their components, such as the working solid state electrodes and reference electrodes, according to the description provided here.

The sweat analyte measurements can be used alone or in combination with other analytes, biomarkers, physiological data, environmental data, user data or population data, and pattern recognition, or informatics, to determine the state, or health state of the organism being measured. The changes in the solid state electrode measurements can be monitored by an algorithm using either the exact measurement levels, or their values in relation to other measurements of the same sensor, or in relation to measurements of other biomarkers, or sensors, or data such as population data or user data. The drift or change in sensor or solid state electrode measurements over time, or the accumulation of data points over time, can be analyzed to assist in the determination of the analyte concentration. Z scores, normalization, and other data modeling approaches can be used in the analysis. This information can be used either to show the levels of the analyte, or to be used to recognize the existence of (in the case of cancer, for example), state of, or tendency toward a condition. The measurements of the electrodes themselves that make up a sensor or multiple sensors, can be compared to each other to assist in analysis. The differences between electrodes, such as the differential between the working and reference electrodes, can be used to determine an analyte level. In one embodiment, one of the electrodes is grounded out in relation to the other electrode, to obtain a single differential value for comparison. In other cases, electrode measurements may be used separately in the analysis. Additional electrodes may be used in the analysis of the level of the analyte, by providing an additional measurement value of the same analyte; selecting for another analyte; or for use in detecting other influences that may impact the measurements of the electrodes in general. For example, what would normally be a two-electrode system may utilize additional electrodes, such as a counter electrode to monitor fluctuations in current. Further, the counter electrode can help in drawing the current away from the reference electrode, which helps in conserving the composition of the reference electrode. The data model works with a group of these inputs where some inputs may influence the analysis of other inputs in order to approximate the biomarker level (e.g. temperature influencing pH levels, fluctuations in current influencing analyte levels).

The data from the solid state electrodes and sensor can be collected and analyzed via an electronic device or sent over a network to an external device, such as a mobile phone, for analysis, or manually inputted into software for analysis.

While many of the examples involve a two-electrode system (working and reference solid state electrodes), this is for exemplary purposes and the claims should not be so limited, and other embodiments may include an electrode system that uses any number of electrodes. For example, the biosensor for the ions can have a working electrode, a reference electrode, and a counter electrode. In an embodiment, the electrode system comprises multiple electrochemical cells with one or more than one, such as 1 to 5, or 1 to 15, or 1 to 25 electrodes for each cell. The electrode system can have multiple electrochemical cells that share electrodes. In an embodiment, the electrode system can have an array of electrochemical cells each with their own working electrodes, and a shared reference electrode wherein the cells can sense separate analytes in the same sample that is in contact with the cells and electrodes.

### EXAMPLES

### Physical Deposition

The general steps in a physical deposition process to prepare a solid state electrode include: deposition of nanoparticles with a compound of the metal used in the metal electrode onto the metal electrode surface to make the solid state electrode; chemically modifying the nanoparticles to reduce the surface charge, then depositing the nanoparticles together with the compound of the metal used in the metal electrode onto the metal electrode surface to make the solid state electrode; and attachment of proteins, such as strongly adhesive proteins, one or more polymers, such as amyloid type nanofibrils, or PVB, to act as a diffusion barrier. In some embodiments, nanocomposites of proteins, and nanoparticles, such as titanium dioxide (TiO2) nanoparticles, can be used to protect the solid state electrode. Because some sample mediums or device setups can be abrasive to the sensor, such as soil samples or where the sensor is exposed to friction or placed in direct contact with an external surface, it may be desirable to protect the solid state electrode with “tough” nanocomposites of proteins and nanoparticles. Without protection, the soil particles or friction, for example, can erode the solid state electrodes. Solid state electrode erosion means that the device would not be as durable as a solid state electrode that did not erode. Protection of the solid state electrode can also be used for other analytes that may contain particles that can damage the electrode, including drug suspensions and environmental water samples, for example. For soil pH sensing, where soil can be abrasive, for example, nanocomposites of proteins, such as mussel adhesive proteins, and nanoparticles, such as titanium dioxide (TiO2) nanoparticles, can be used to protect the solid state electrode. Without protection, the soil particles can erode the solid state electrodes. Solid state electrode erosion means that the device would not be as durable as a solid state electrode that did not erode.

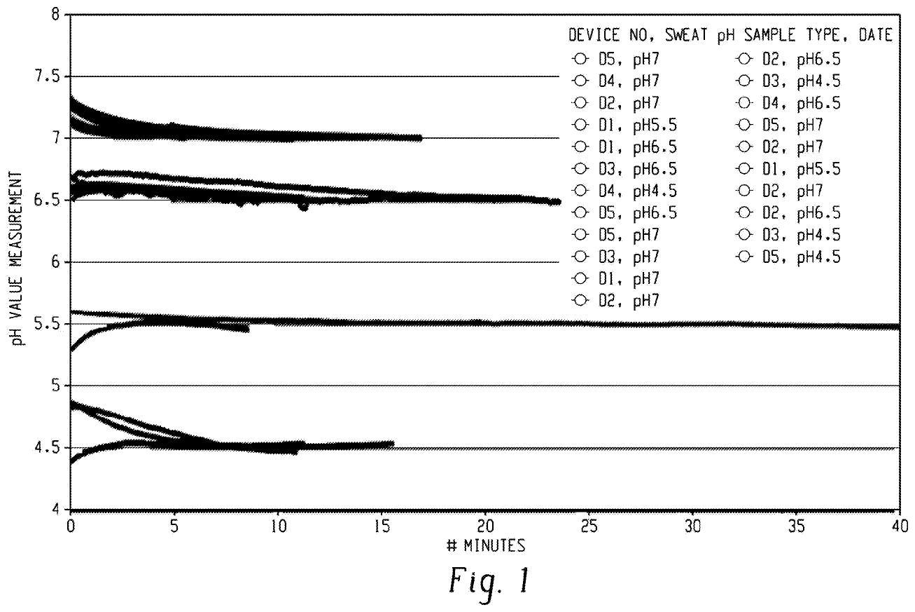

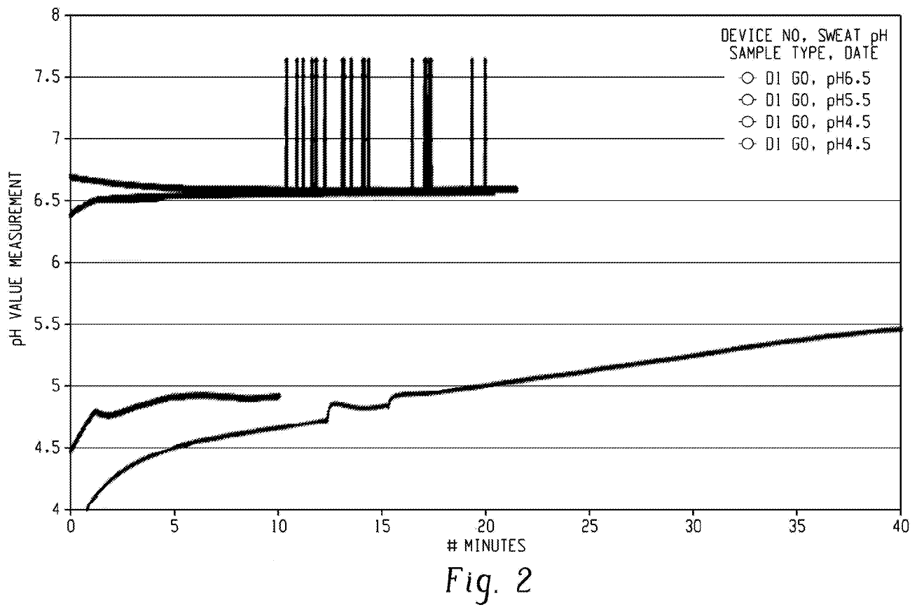

Nanocomposites formed by combining a compound of a metal used in the electrode and nanoparticles show good stability. An experiment was performed. A 65 microliter (μl) drop of solution was applied to cover the solid state reference electrode and the working electrode, the run was started by switching on a power unit powered by a battery. The runs were conducted for periods ranging between 15 minutes and 60 minutes each day. The devices were tested for a maximum of 1 hour each day due to the high number of sensors needing tested. The following observations were made based on the experimental results: The solid state electrodes are non polarizable. That is the potential was maintained for each specific buffer, when buffers of different pH's and deionized (DI) water were rotated as samples in the tests. Various buffer solutions were used that contained different kinds of ions, including chlorides, phosphates, oxalates and sulfates without significant changes in potential. After conducting tests on the sensors, a data sampling of which is shown in Table 1, a side-by-side comparison was made to an electrode using Graphene Oxide (GO). The GO approach (Table 1) showed rapid disintegration of the electrodes in the first 3 tests and difficulty reaching equilibrium. The sensor described here, however, demonstrated that even over a few months, the sensors were able to repeatedly detect the correct pH level within 0.02 of the target pH Value without the need to modify or calibrate the sensors in-between tests. The average distance from the target pH was 0.02, a standard deviation of +/−0.02, and mean mV change of 1.44. These sensors show sensitivities similar to high-resolution commercial macro pH meters. Many standard commercial pH meters and test strips only have a standard deviation of +/−0.1 to 1 pH unit. Between pH 4 and pH 7, a solid state reference electrode made as described here exhibited stable values within 2 mV over 5 months of regular testing at 1 hour test runs without showing significant drifts in the potentials measured. Table 1 and FIG. 1 show stable solid state electrodes made by the methods described here. FIG. 2 shows the pH stability of a graphene oxide sensor device without the modifications to the reference electrode as described herein and tested using the same conditions as the sensor devices as described in FIG. 1. Data shows that the electrodes can maintain excellent stability when tested in different conditions. The solid state electrodes show sensitivities of down to a concentration of 10⁻⁴ for the ions that were analyzed. which is within the detection range of numerous analytes in sweat. The table data is based on experiments run for at least 5 months where sensors were tested repeatedly and without modification or calibration.

TABLE 1

| | | | | | Standard | | Standard | | |
|---|---|---|---|---|---|---|---|---|---|
| | Test | | | Sample | Deviation | Mean | Deviation | Mean | Distance |
| | Name | Device# | Date | Value | (pH) | (pH) | (mV) | (mV) | From Target |
| Described | D3, | 3 | Month | 4.5 | 0.01 | 4.517 | 0.464 | 198.19 | 0.016530612 |
| Approach | pH4.5 | | 3 | | | | | | |
| | D4, | 4 | Month | 4.5 | 0.01 | 4.524 | 0.527 | 197.81 | 0.024285714 |
| | pH4.5 | | 3 | | | | | | |
| | D5, | 5 | Month | 4.5 | 0.04 | 4.509 | 2.040 | 198.56 | 0.008979592 |
| | pH4.5 | | 3 | | | | | | |
| | D1, | 1 | Month | 5.5 | 0.04 | 5.466 | 1.709 | 151.69 | 0.034489796 |
| | pH5.5 | | 3 | | | | | | |
| | D1, | 1 | Month | 5.5 | 0.02 | 5.511 | 0.648 | 149.63 | 0.010571429 |
| | pH5.5 | | 3 | | | | | | |
| | D1, | 1 | Month | 6.5 | 0.02 | 6.534 | 2.482 | 111.56 | 0.03368586 |
| | pH6.5 | | 3 | | | | | | |
| | D2, | 2 | Month | 6.5 | 0.02 | 6.561 | 1.919 | 108.75 | 0.061202507 |
| | pH6.5 | | 3 | | | | | | |
| | D2, | 2 | Month | 6.5 | 0.02 | 6.534 | 2.230 | 111.56 | 0.03368586 |
| | pH6.5 | | 3 | | | | | | |
| | D3, | 3 | Month | 6.5 | 0.02 | 6.530 | 1.704 | 111.94 | 0.029964747 |
| | pH6.5 | | 3 | | | | | | |
| | D3, | 3 | Month | 6.5 | 0.01 | 6.512 | 1.085 | 113.81 | 0.011652957 |
| | pH6.5 | | 3 | | | | | | |
| | D4, | 4 | Month | 6.5 | 0.02 | 6.540 | 2.354 | 110.81 | 0.040099882 |
| | pH6.5 | | 3 | | | | | | |
| | D5, | 5 | Month | 6.5 | 0.03 | 6.547 | 2.918 | 110.25 | 0.046513905 |
| | pH6.5 | | 3 | | | | | | |
| | D1, pH7 | 1 | Month | 7 | 0.02 | 7.037 | 1.581 | 60.19 | 0.036721504 |
| | | | 1 | | | | | | |
| | D2, pH7 | 2 | Month | 7 | 0.01 | 7.017 | 1.318 | 62.25 | 0.016549158 |
| | | | 1 | | | | | | |
| | D2, pH7 | 2 | Month | 7 | 0.03 | 7.024 | 2.568 | 61.5 | 0.023893459 |
| | | | 1 | | | | | | |
| | D2, pH7 | 2 | Month | 7 | 0.02 | 7.029 | 2.100 | 60.94 | 0.029377203 |
| | | | 1 | | | | | | |
| | D2, pH7 | 2 | Month | 7 | 0.01 | 7.028 | 1.376 | 61.13 | 0.027516647 |
| | | | 1 | | | | | | |
| | D3, pH7 | 3 | Month | 7 | 0.00 | 7.002 | 0.165 | 63.75 | 0.001860556 |
| | | | 1 | | | | | | |
| | D4, pH7 | 4 | Month | 7 | 0.00 | 7.002 | 0.378 | 63.75 | 0.001860556 |
| | | | 1 | | | | | | |
| | D5, pH7 | 5 | Month | 7 | 0.01 | 7.007 | 0.576 | 63.19 | 0.007344301 |
| | | | 1 | | | | | | |
| | D5, pH7 | 5 | Month | 7 | 0.01 | 7.013 | 0.847 | 62.63 | 0.012828045 |
| | | | 1 | | | | | | |
| | D5, pH7 | 5 | Month | 7 | 0.01 | 6.998 | 0.629 | 64.12 | 0.001762632 |
| | | | 1 | | | | | | |
| AVERAGE | | | | | 0.02 | | 1.44 | | 0.02 |
| Graphene | D1 GO, | 1GO | Day 1 | 4.5 | 0.02 | 4.914 | 0.940 | 178.69 | 0.414489796 |
| Oxide | pH4.5 | | | | | | | | |
| | D1 GO, | 1GO | Day 3 | 4.5 | 0.30 | 5.048 | 14.560 | 172.13 | 0.548367347 |
| | pH4.5 | | | | | | | | |
| | D2 GO, | 2GO | Day 3 | 4.5 | 0.06 | 5.026 | 2.732 | 173.25 | 0.525510204 |
| | pH4.5 | | | | | | | | |
| | D1 GO, | 1GO | Day 1 | 5.5 | 0.12 | 6.554 | 12.489 | 109.5 | 1.053858206 |
| | pH5.5 | | | | | | | | |
| | D1 GO, | 1GO | Day 1 | 6.5 | 0.01 | 6.583 | 0.732 | 106.5 | 0.083235409 |
| | pH6.5 | | | | | | | | |
| AVERAGE | | | | | 0.10 | | 6.29 | | 0.53 |

Another embodiment for attaching nanoparticles to make stable solid state reference electrodes involves using modified nanoparticles. In this approach, the nanoparticles are first modified with hydrophobic compounds containing an amine group and a thiol group via covalent bonding. The nanoparticles can also be modified with a hydrophobic compound containing a carboxyl group and a thiol group. Thiol containing compounds are known to interact strongly with metal atoms. The surface modification of the nanoparticles reduces the surface charge. The thiol containing compounds can be attached to the nanoparticles using the amine group by well-known chemical reactions. The modified nanoparticles can be attached to the compound of the metal used in the metal electrode using physical deposition in combination with an oxidizing agent. The modified nanoparticles interact strongly with the metal atoms in the compound of the metal used in the electrode via the thiol groups, creating a robust structure. Further, these nanoparticles reduce the solubility of the compound of the metal used in the electrode. The reduction in the solubility of the compound of the metal used in the electrode slows down the loss of it from the reference solid state electrode.

Another alternative approach to stabilize the solid state electrode is attachment of proteins, and/or polymers to the electrodes. The proteins are mixed with an oxidizing agent and nanoparticles and physically attached to the metal electrode to produce composites that strongly stick to the surface. These proteins are also used as thin layers on top of electrodes modified as described above. In some cases, an oxidant can be used to accelerate the crosslinking of the proteins. In some cases, it is important for the proteins to be cross-linked so that they can effectively encapsulate the electrode. Crosslinking is believed to impart physical stability to the protein. Polymers or peptides can be mixed with an oxidizing agent and nanoparticles in the same way as the proteins to produce nanocomposite reference solid state electrodes. A top layer of polymers can also be deposited on top of the electrode to act as a diffusion barrier, similarly to the proteins.

The following polymers can be used, which have been shown to adhere strongly to surfaces, such as polyvinyl butyral (PVB), however, any polymer that exhibits strongly binding characteristics can be used.

The following proteins or combinations thereof can be used, which have been shown to adhere strongly to surfaces, such as amyloid fibrils, amyloid nanofibrils, adhesive proteins, mussel proteins.

### Electrochemical Deposition

The general steps in an electrochemical deposition process to prepare a solid state electrode include: electrochemical deposition of nanoparticles with a compound of a metal used in the electrode onto a metal electrode surface, chemically modifying the nanoparticles to reduce the surface charge, then electrochemically depositing the nanoparticles together with the compound of a metal used in the electrode onto the electrode surface; and electrochemical attachment of proteins and/or polymers to act as a diffusion barrier.

For electrochemical deposition of compound of a metal used in the electrode onto the metal surface of the electrode, 1 M or 2 M, for example, of an acid is used and a voltage or current applied. As an example a current of 20 μA for 1 minute or 2 minutes is applied. The applied voltage produces a coating of a compound of the metal used in the metal electrode on the metal electrode surface. Electrochemical deposition of nanoparticles from solution is performed over the deposited layer of the compound of the metal used in the metal electrode. The nanoparticles attach to the layer of the compound of the metal used in the metal electrode via redox processes. A portion of the surface comprising the compound of the metal used in the metal electrode, or the entire surface of the compound of the metal used in the metal electrode can be coated with nanoparticles, depending on the amount of component in solution, the charge, and other factors known in the art.

Besides covering the layer of the compound of the metal used in the electrode with nanoparticles, mixed composites of the compound of the metal used in the electrode with nanoparticles can be made. An acid can be mixed with nanoparticles in a solution, and the mixed solution can be applied to the electrode. Next, a potential is applied to the electrode to electrochemically attach the compound of the metal used in the electrode-nanoparticles composites to the electrode surface. Because of the electrochemical processes, the compound of the metal used in the electrode and the nanoparticles are chemically bound which produces a robust electrode.

Nanoparticles that are first chemically modified then deposited together with the compound of the metal used in the electrode, can be used, as described above. Hydrophobic compounds containing an anime group, a thiol group, and an aromatic group are used in an example. Next, the modified nanoparticles are mixed with acid to form a solution. The solution mixture is applied to the electrode surface and a potential is applied to the electrode. The application of the potential electrochemically deposits the nanoparticles together with the compound of the metal used in the electrode on the electrode surface. The thiol modified nanoparticles form strong bonds with the metal atoms in the compound of the metal used in the electrode during the electrochemical process. The strong bonding produces a stable structure. Further, the aromatic hydrophobic groups attached to the nanoparticles reduce the solubility of the compound of the metal used in the electrode. Although Applicant does not wish to be bound by any theory provided, reducing the solubility of the compound of the metal used in the electrode is known to be useful in slowing down its loss from the electrode during operation.

Similar to the physical deposition methods, proteins can be attached to the electrode prepared using electrochemical methods. Electrodes prepared using the approaches above are modified on the surface using the proteins. A small drop of a protein solution, in many cases the solution is water, is applied to the electrodes. Next, a drop of oxidant is added to the electrodes. Depending on the type of protein, the electrodes are left for 30 minutes or overnight, for example, to allow the oxidant to crosslink the protein. The crosslinked protein layer on top of the electrode reduces loss of the compound of the metal used in the electrode.

Polymers such as PVB can be attached to the electrode by drop casting a solution of PVB in organic solvent.

In a particular example, electrochemical deposition of a compound of the metal used in the metal electrode in the presence of the protein is performed. The protein is mixed with acid and the solution placed on the electrode. Application of voltage or current deposits the compound of the metal used in the electrode together with the protein. Another approach is to mix the protein with nanoparticles, and acid. Another approach is to mix the protein with nanoparticles. Applying a voltage deposits a nanocomposite of a compound of the metal used in the metal electrode, nanoparticles, and one or more proteins.

### Surface Analysis

The solid state electrodes produced by the methods outlined above are characterized using microscopic imaging and spectroscopy. Microscopic techniques for imaging include atomic force microscopy (AFM) and scanning electron microscopy (SEM). AFM provides images of structures that are as small as 2 nanometers. The detail in AFM imaging provides information on the very small nanocomposites that are produced during the deposition. On the other hand, SEM provides information on the micro-sized structures that are produced and on the distribution of the nanostructures. Spectroscopy is used to identify the chemical groups that are on the surface of the solid state electrode. While the materials that are attached to the electrode are known, chemical changes may occur during the deposition, resulting in the alteration of the chemistry of these materials. Spectroscopic techniques include infra-red spectroscopy (IR), x-ray photoelectron spectroscopy (XPS), Raman spectroscopy, and fluorescence spectroscopy.

### Biosensor

The solid state electrodes described herein can be used in a biosensor. Some factors that influence the usefulness of biosensors include stability, sensitivity/resolution, fluid transport, biocompatible materials, duration of use, durability, resistance to external influencing factors, and manufacturing potential.

Manufacturing processes that use nanofabrication or microfabrication processes to create functional sensors can be used to deposit the materials onto the electrodes.

The methods described herein ensure reproducibility of the composition of the prepared electrodes.

The solid state electrodes used in a sensor can be used in microfluidic chips or screen printed electronics and sensors.

In order to detect a small change in pH, such as 0.05 pH units, for example, a sensitive working electrode is needed which can detect potential differences between 3mV -5 mV, for example. To provide a sufficiently sensitive and reproducible working electrode, the proper thickness for each material should be used. The thickness is important in determining the difference in concentration between the surface of the membrane and the bulk sample. Spincoating is one technique used to make membranes of a specified thickness for sensitivity testing. The spincoating procedure can be followed by vacuuming of the spin coated layer. The vacuuming procedure may remove bubbles that form on the membrane. The bubbles that can form during spincoating can be considered as defects on the membrane which can reduce sensitivity. The membranes are imaged using microscopic imaging to obtain information on the thickness of the membranes after spincoating. The presence of structural defects on the membranes are also determined using microscopic imaging. If the membrane has any defects, then the sensitivity may be lower than desired. Defects may result in bubbles and inflow of ions other than the desired ones.

Other embodiments include using metal oxide instead of a membrane, such as an ion selective membrane. Metal oxides, such as pH sensors, present technical challenges because their processing may include heating to around 450° C. after sol-gel synthesis. This means that the sensor materials should be able to withstand these temperatures for this approach to be used. Metal oxides that can be used for pH sensing include palladium oxide, platinum oxide, ruthenium oxide and zinc oxide. Some oxides such as zinc oxide or platinum oxide can be created in-situ on the working electrode by applying a current to the working electrode for a few seconds. The sensing of pH using metal oxide films on the working electrode is based on the protonation and deprotonation of the metal oxide films as the pH is varied. The protonation and deprotonation of the metal oxide films results in a change of the electrode potential.

The sensors can be printed with stretchable inks. For example, those made by DuPont.

Fluid manipulation is a factor for keeping a good working environment for the solid state electrodes. Since the devices can be used for intermittent or continuous monitoring of biofluids such as sweat, it is expected that some of these fluids may deposit salts and other compounds on the electrode area, resulting in poor electrode performance. A flushing system to flush out ions, unwanted salts, and other compounds can be used so that the device can continue working properly. The flushing system can be integrated with the device, and in an example, the flushing system can be triggered by the presence of high levels of electrolytes that may damage the solid state electrodes. The flushing system can be arranged so as to provide a flow of liquid, such as deionized water, over an electrode.

The bio sensor should be durable, in part because the biosensor can be worn, or attached to the skin or exposed for long periods of time. Biocompatible materials with waterproofing for liquid sensitive components can be used to improve durability.

In order to have a stable chemically selective working electrode, one approach is to use hydrophobic conducting polymers which are deposited on the working electrode before attaching the selective membrane, so that the conducting polymer can act as a uniform interlayer. Deposition of the polymer can be done by spincoating, drop casting, or electropolymerization. The polymer can be synthesized in-situ by electropolymerization of the monomer. The conducting polymer can exclude water from the interface, resulting in a stable potential. Polymers can include any conducting polymers, such as poly(3,4-diethylenedioxythiophene) (PEDOT), polypyrrole, polyaniline, polythiophene, polyoctylthiophene (POT), P3HT, and polytetrafluoroethylene. These polymers are well known for their versatility.

Besides conducting polymers, in an embodiment, the deposition of a layer of nanoparticles on the working electrode is performed before attaching the selective membrane. The nanoparticles can be physically or electrochemically deposited. Physical deposition can be done by drop casting the polymer onto the electrode from solution. Electrochemical deposition can be achieved by drop casting a solution of nanoparticles to the electrode and then applying a potential to the electrode. Application of potential can result in the oxidation of the nanoparticles and the nanoparticles become attached to the working electrode. Nanoparticles can also be produced in situ from a precursor in solution, during an electrochemical process. By applying a current to a precursor in solution, such as zinc chloride solution, one can produce zinc oxide nanoparticles in situ on the electrode. This produces a hydrophobic interlayer. A hydrophobic layer can exclude water from the electrode, resulting in a stable potential of the working electrode.

A solution can be but is not limited to an aqueous or organic (e.g. ethanol, propanol, acetyl nitrile, methanol) solution, for example.

Another option for the interlayer will be the use of nanoparticles, such as gold nanoparticles, modified with hydrophobic ligands. Particles sized between 5 nm and 12 nm can be used, for example. One method of deposition the nanoparticles is by spin coating from organic solvent. The hydrophobic ligands also exclude water collection at the interlayer. Hydrophobic ligands can include Thiophenol, 2-Napthalenethiol, 9-Anthracenethiol.

Another option for the interlayer is the use of a hydrophobic metal oxide layer which can be produced by electrochemical deposition on the working electrode from a solution of the compound of a metal, where the metal can be zinc, copper, nickel, platinum, cobalt, tantalum, or other metals that can produce an oxide layer. For example, a zinc chloride solution can produce a zinc oxide layer on the working electrode.

A combination of multiple types of nanoparticles can also be used to create the interlayer film.

Detection can be done using potentiometric sensing or amperometric sensing depending on the species being detected, for any of the devices described herein. Amperometric sensing for example is preferred when using biorecognition elements or redox processes, such as when using glucose oxidase.

The compositions, methods, articles, and other aspects are further described by the Embodiments below.

### Embodiments

Embodiment 1: A solid state reference electrode comprising: a metal electrode having a surface; a nanocomposite coated on at least a portion of the surface, the nanocomposite comprising a compound of the metal used in the metal electrode, and nanoparticles, a protein, a polymer, or a combination comprising at least one of the foregoing; wherein when the solid state reference electrode is in electrical connection with a working electrode and a conductive substance, the electrode can detect a change in an analyte), and the potential of the electrode is stable to within 5 millivolts, preferably within 3 millivolts over a period of 20 minutes.

Embodiment 2: The solid state reference electrode of Embodiment 1, wherein the conductive substance is a gel.

Embodiment 2A: The solid state reference electrode of Embodiment 1, wherein the conductive substance is a fluid.

Embodiment 3: The solid state reference electrode of Embodiment 1, wherein the conductive substance is a body fluid.

Embodiment 4: The solid state reference electrode of Embodiment 1, 2 or 3, wherein the solid state reference electrode can detect a change in pH of less than or equal to 0.5 pH units, and in one embodiment less than or equal to 0.05 pH units.

Embodiment 5: The solid state reference electrode of any one or more of Embodiments 1-4, wherein the nanoparticles are gold nanoparticles.

Embodiment 6: The solid state reference electrode of any one or more of Embodiments 1-4, wherein the nanoparticles are silver nanoparticles.

Embodiment 7: The solid state reference electrode of any one or more of Embodiments 1-4, wherein the nanoparticles are copper nanoparticles.

Embodiment 8: The solid state reference electrode of any one or more of Embodiments 1-4, wherein the nanoparticles are carbon nanoparticles.

Embodiment 9: The solid state reference electrode of any one or more of Embodiments 1-4, wherein the nanoparticles are zinc oxide nanoparticles

Embodiment 10: The solid state reference electrode of any one or more of Embodiments 1-4, wherein the nanoparticles are spherical carbon nanoparticles.

Embodiment 11: The solid state reference electrode of any one or more of Embodiments 1-4, wherein the nanoparticles are graphene oxide.

Embodiment 12: The solid state reference electrode of any one or more of Embodiments 1-4, wherein the nanoparticles are carbon nanotubes.

Embodiment 13: The solid state reference electrode of any one or more of Embodiments 1-4, wherein the nanoparticles are quantum dots.

Embodiment 14: The solid state reference electrode of any one or more of Embodiments 1-4, wherein the nanoparticles are diamond nanoparticles.

Embodiment 15: The solid state reference electrode of any one or more of Embodiments 1-4, wherein the nanoparticles are graphene quantum dots.

Embodiment 16: The solid state reference electrode of any one or more of Embodiments 1-4, wherein the nanoparticles are titanium oxide nanoparticles.

Embodiment 17: The solid state reference electrode of any one or more of Embodiments 1-4, wherein the nanoparticles are silicon oxide nanoparticles.

Embodiment 18: The solid state reference electrode of any one or more of Embodiments 1-4, wherein the nanoparticles are carbon quantum dots.

Embodiment 19: The solid state reference electrode of any one or more of Embodiments 1-4, wherein the nanoparticles are nanoclusters.

Embodiment 20: The solid state reference electrode of any one or more of Embodiments 1-4, wherein the nanoparticles are gold nanoclusters.

Embodiment 21: The solid state reference electrode of any one or more of Embodiments 1-4, wherein the nanoparticles are silver nanoclusters.

Embodiment 22: The solid state reference electrode of any one or more of Embodiments 1-4, wherein the nanoparticles are europium oxide nanoparticles.

Embodiment 23: The solid state reference electrode of any one or more of Embodiments 1-4, wherein the nanoparticles are fullerenes.

Embodiment 24: The solid state reference electrode of any one or more of Embodiments 1-4, wherein the nanoparticles are iron oxide nanoparticles.

Embodiment 25: The solid state reference electrode of any one or more of Embodiments 1-24, wherein the nanoparticles are modified, wherein a hydrophobic compound comprising an amine group and a thiol group is covalently bonded to the nanoparticles.

Embodiment 26: The solid state electrode of Embodiment 25, wherein the hydrophobic compound is 4-aminothiophenol, 5-amino-2-mercaptobenzimidazole, 5-carboxy-2-mercaptobenzimidazole, Thiophenol, 2-Napthalenethiol, or 9-Anthracenethiol.

Embodiment 27: The solid state electrode of any one or more of Embodiments 1-26, wherein the nanoparticles have an average diameter of less than or equal to 200 nanometers.

Embodiment 28: A method of making a solid state reference electrode, comprising: providing a metal electrode having a surface; attaching a nanocomposite comprising a compound of the metal used in the metal electrode, and nanoparticles, a polymer or polymers, a protein or proteins, or a combination comprising at least one of the foregoing, onto at least a portion of the metal surface.

Embodiment 29: The method of Embodiment 28, wherein attaching is physical deposition, chemical deposition or electrochemical deposition.

Embodiment 30: The method of Embodiments 28 or 29, further comprising: applying a voltage to the surface in an acid solution, forming a coated surface of a compound of the metal used in the metal electrode; and electrochemically depositing the nanocomposite onto the compound of the metal used in the metal electrode coated surface.

Embodiment 31: The method of any one or more of Embodiments 28-30, wherein the nanocomposite comprises nanoparticles.

Embodiment 32: The method of any one or more of Embodiments 28-31, wherein the nanoparticles are gold nanoparticles.

Embodiment 33: The method of any one or more of Embodiments 28-31, wherein the nanoparticles are silver nanoparticles.

Embodiment 34: The method of any one or more of Embodiments 28-31, wherein the nanoparticles are copper nanoparticles.

Embodiment 35: The method of any one or more of Embodiments 28-31, wherein the nanoparticles are carbon nanoparticles.

Embodiment 36: The method of any one or more of Embodiments 28-31, wherein the nanoparticles are zinc oxide nanoparticles.

Embodiment 37: The method of any one or more of Embodiments 28-31, wherein the nanoparticles are spherical carbon nanoparticles.

Embodiment 38: The method of any one or more of Embodiments 28-31, wherein the nanoparticles are fullerenes.

Embodiment 39: The method of any one or more of Embodiments 28-31, wherein the nanoparticles are quantum dots.

Embodiment 40: The method of any one or more of Embodiments 28-31, wherein the nanoparticles are graphene oxide.

Embodiment 41: The method of any one or more of Embodiments 28-31, wherein the nanoparticles are carbon nanotubes.

Embodiment 42: The method of any one or more of Embodiments 28-31, wherein the nanoparticles are diamond nanoparticles.

Embodiment 43: The method of any one or more of Embodiments 28-31, wherein the nanoparticles are graphene quantum dots.

Embodiment 44: The method of any one or more of Embodiments 28-31, wherein the nanoparticles are carbon quantum dots.

Embodiment 45: The method of any one or more of Embodiments 28-31, wherein the nanoparticles are titanium oxide nanoparticles.

Embodiment 46: The method of any one or more of Embodiments 28-31, wherein the nanoparticles are silicon oxide nanoparticles.

Embodiment 47: The method of any one or more of Embodiments 28-31, wherein the nanoparticles are gold nanoclusters.

Embodiment 48: The method of any one or more of Embodiments 28-31, wherein the nanoparticles are nanofibers.

Embodiment 49: The method of any one or more of Embodiments 28-31, wherein the nanoparticles are carbon nanofibers.

Embodiment 50: The method of any one or more of Embodiments 28-31, wherein the nanoparticles are silver nanoclusters.

Embodiment 51: The method of any one or more of Embodiments 28-31, wherein the nanoparticles are europium oxide nanoparticles.

Embodiment 52: The method of any one or more of Embodiments 28-31, wherein the nanoparticles are iron oxide nanoparticles.

Embodiment 53: The method of any one or more of Embodiments 28-31, wherein the nanoparticles are nanoclusters.

Embodiment 54: The method of any one or more of Embodiments 1-53, wherein the nanocomposite comprises a compound of a metal used in the electrode and nanoparticles.

Embodiment 55: The method of any one or more of Embodiments 1-54, wherein the nanocomposite comprises mercury chloride and nanoparticles.

Embodiment 56: The method of any one or more of Embodiments 1-54, wherein the nanocomposite comprises copper sulfate and nanoparticles.

Embodiment 57: The method of any one or more of Embodiments 1-54, wherein the nanocomposite comprises silver chloride and nanoparticles.

Embodiment 58: The method of any one or more of Embodiments 1-54, wherein the nanocomposite comprises mercury chloride and metal nanoparticles.

Embodiment 59: The method of any one or more of Embodiments 1-54, wherein the nanocomposite comprises copper sulfate and metal nanoparticles.

Embodiment 60: The method of any one or more of Embodiments 1-54, wherein the nanocomposite comprises silver chloride and metal nanoparticles.

Embodiment 61: The method of any one or more of Embodiments 1-54, wherein the nanocomposite comprises mercury chloride and carbon nanoparticles.

Embodiment 62: The method of any one or more of Embodiments 1-54, wherein the nanocomposite comprises copper sulfate and carbon nanoparticles.

Embodiment 63: The method of any one or more of Embodiments 1-54, wherein the nanocomposite comprises silver chloride and carbon nanoparticles.

Embodiment 64: The method of Embodiment 28, wherein attaching comprises: contacting a solution of acid and nanoparticles with the surface; applying a voltage to the surface, forming a coated surface comprising a composite of the compound of the metal used in the metal electrode-nanoparticle.

Embodiment 65: The method of Embodiment 28, wherein attaching comprises: contacting an aqueous solution of potassium chloride and nanoparticles with the surface; applying a voltage to the surface, forming a mercury chloride-nanoparticle composite coated surface.

Embodiment 66: The method of Embodiment 28, wherein attaching comprises: contacting an aqueous solution of potassium chloride and nanoparticles with the surface; applying a voltage to the surface, forming a silver chloride-nanoparticle composite coated surface.

Embodiment 67: The method of Embodiment 28, wherein the nanocomposite comprises a protein and nanoparticles, and the method further comprises mixing the protein and nanoparticles with oxidizing agent prior to attaching the nanocomposite onto the surface.

Embodiment 68: The method of Embodiments 28, wherein the nanocomposite comprises nanoparticles, and wherein attaching comprises: mixing the nanoparticles with an oxidizing agent in solution, to form a solution of oxidizing agent and nanoparticles; and applying the solution of oxidizing agent and nanoparticles to the surface to deposit a composite of the compound of the metal used in the metal electrode and nanoparticle.

Embodiment 69: The method of Embodiments 67 or 68, wherein the oxidizing agent is permanganate dichromate, iron(III), perchlorate, periodate, hydrogen peroxide, chlorate, chromate or iodate.

Embodiment 70: The method of Embodiment 28, wherein the nanocomposite comprises an adhesive protein and nanoparticles, and the method further comprises mixing the adhesive protein and nanoparticles with an oxidizing agent prior to attaching the nanocomposite onto the surface.

Embodiment 71: The method of Embodiment 28, wherein the nanocomposite comprises an adhesive protein and carbon nanoparticles, and the method further comprises mixing the adhesive protein and carbon nanoparticles with an oxidizing agent prior to attaching the nanocomposite onto the surface.

Embodiment 72: The method of Embodiment 28, wherein the nanocomposite comprises an adhesive protein and metal nanoparticles, and the method further comprises mixing the adhesive protein and metal nanoparticles with an oxidizing agent prior to attaching the nanocomposite onto the surface.

Embodiment 73: The method of Embodiment 28, wherein the nanocomposite comprises amyloid type nanofibrils and nanoparticles, and the method further comprises mixing the amyloid type nanofibrils and nanoparticles with an oxidizing agent prior to attaching the nanocomposite onto the surface.

Embodiment 74: The method of Embodiment 28, wherein the nanocomposite comprises amyloid type nanofibrils and carbon nanoparticles, and the method further comprises mixing the amyloid type nanofibrils and carbon nanoparticles with an oxidizing agent prior to attaching the nanocomposite onto the surface.

Embodiment 75: The method of Embodiment 28, wherein the nanocomposite comprises amyloid type nanofibrils and metal nanoparticles, and the method further comprises mixing the amyloid type nanofibrils and metal nanoparticles with an oxidizing agent prior to attaching the nanocomposite onto the surface.

Embodiment 76: The method of Embodiment 28, wherein the nanocomposite comprises a polymer and nanoparticles.

Embodiment 77: The method of Embodiment 28, wherein the nanocomposite comprises PVB and metal nanoparticles.

Embodiment 78: The method of Embodiment 28, wherein the nanocomposite comprises PVB and carbon nanoparticles.

Embodiment 79: The method of Embodiment 28, wherein the nanoparticles are modified nanoparticles, wherein a hydrophobic compound comprising an amine group and a thiol group is covalently bonded to the nanoparticles.

Embodiment 80: The method of Embodiment 28, wherein the nanoparticles are modified carbon nanoparticles, wherein a hydrophobic compound comprising an amine group and a thiol group is covalently bonded to the carbon nanoparticles.

Embodiment 81: The method of Embodiment 28, wherein the nanoparticles are modified metal nanoparticles, wherein a hydrophobic compound comprising an amine group and a thiol group is covalently bonded to the metal nanoparticles.

Embodiment 82: A biosensor for determining a parameter of a conductive substance, comprising: a substrate having a top surface and a bottom surface; a working electrode comprising a selective membrane or a metal oxide, disposed on the top surface of the substrate; a reference electrode comprising the solid state electrode of one or more of Embodiments 1 to 27, disposed on the top surface of the substrate, wherein the working electrode and reference electrode are electrically coupled when in contact with a conductive substance.

Embodiment 83: A biosensor for determining a parameter of a conductive substance, comprising: a substrate having a surface; a working electrode comprising a selective membrane or a metal oxide, disposed on the surface of the substrate; a reference electrode comprising the solid state electrode of one or more of Embodiments 1 to 27, disposed on the surface of the substrate, wherein the working electrode and reference electrode are electrically coupled when in contact with a conductive substance.

Embodiment 84: The method of any one or more of Embodiments 1 to 27, wherein the conductive substance is a fluid.

Embodiment 85: The method of any one or more of Embodiments 1-27, wherein the conductive substance is a gel.

Embodiment 86: The method of any one or more of Embodiments 1-27, wherein the conductive substance is a metal.

Embodiment 87: The method of any one or more of Embodiments 1-27, wherein the conductive substance is an organic conductive material.

Embodiment 88: A biosensor for determining a parameter of a fluid, comprising: a substrate having a top surface and a bottom surface; a working electrode comprising an ion-selective membrane or a metal oxide, disposed on the top surface of the substrate; a reference electrode comprising the solid state electrode of one or more of Embodiments 1 to 27, disposed on the top surface of the substrate, wherein the working electrode and reference electrode are electrically coupled when in contact with a fluid.

Embodiment 89: A biosensor for determining a parameter of a fluid, comprising: a substrate having a top surface and a bottom surface; a working electrode comprising an ion-selective membrane or a metal oxide, disposed on the bottom surface of the substrate; a reference electrode comprising the solid state electrode of one or more of Embodiments 1 to 27, disposed on the bottom surface of the substrate, wherein the working electrode and reference electrode are electrically coupled when in contact with a fluid.

Embodiment 90: The biosensor or electrode of any one or more of Embodiments 1-27, 82, or 83, wherein the fluid is a body fluid, and wherein the biosensor can be attached to the skin of a human or mammal through the bottom surface of the substrate.

Embodiment 91: The biosensor or electrode of any one or more of Embodiments 1-27, 82, or 83, wherein the fluid is a body fluid, and wherein the biosensor can be attached to the skin of a human or mammal through the top surface of the substrate.

Embodiment 92: The biosensor or electrode of any one or more of Embodiments 1-27, 82, or 83, comprising multiple sensors to detect multiple analytes, which can be used for determining the individual level of an analyte, or used for determining the level of another analyte, (for example a pH sensor and a chloride sensor, where the changes in pH can be used to determine the changes in chloride).

Embodiment 93: The biosensor or electrode of any one or more of Embodiments 1-27, 82, or 83, wherein the fluid is a body fluid and the parameter of the fluid is the level of H+, Na+, Mg2+, NO3−, K+, NH4+, Ca2+, Cl−, testosterone, follicle stimulating hormone (FSH), estrogen, progesterone, androstenedione, beta-human chorionic gonadotrophin (hCG), DNA, RNA, proteins, cytokines, compounds, glucose, xenometabolites, opiates, amphetamines, alcohols, or enzymes.

Embodiment 94: The biosensor or electrode of any one or more of Embodiments 1-27, 82, or 83, wherein the fluid is sweat.

Embodiment 95: The biosensor or electrode of any one or more of Embodiments 1-27, 82, or 83, wherein the fluid is urine.

Embodiment 96: The biosensor or electrode of any one or more of Embodiments 1-27, 82, or 83, wherein the fluid is blood.

Embodiment 97: The biosensor or electrode of any one or more of Embodiments 1-27, 82, or 83, wherein the fluid is saliva.

Embodiment 98: The biosensor or electrode of any one or more of Embodiments 1-27, 82, or 83, wherein the biosensor is between 0.5 and 10 millimeters thick.

Embodiment 99: The biosensor or electrode of any one or more of Embodiments 1-27, 82, or 83, further comprising a flushing system to reduce the concentration of ions or other analytes on one or more electrodes.

Embodiment 100: The biosensor or electrode of any one or more of Embodiments 1-27, 82, or 83, wherein the electrode is deposited on the substrate by screen printing, roll-to-roll printing, aerosol deposition, inkjet printing, thin film deposition, or electroplating.

Embodiment 101: The biosensor or electrode of any one or more of Embodiments 1-27, 82, or 83, wherein the reference electrode is deposited on the substrate by screen printing, roll-to-roll printing, aerosol deposition, inkjet printing, thin film deposition, or electroplating.

Embodiment 102: The biosensor or electrode of any one or more of Embodiments 1-27, 82, or 83, wherein the working electrode is deposited on the substrate by screen printing, roll-to-roll printing, aerosol deposition, inkjet printing, thin film deposition, or electroplating.

Embodiment 103: A sensor for determining a parameter of a conductive substance, comprising: a substrate having a top surface and a bottom surface; a working electrode comprising an selective membrane or a metal oxide, disposed on the top surface of the substrate; a reference electrode comprising the electrode of one or more of Embodiments 1 to 27, or 82 or 83, disposed on the top surface of the substrate, wherein the working electrode and reference electrode are electrically coupled when in contact with a conductive substance, where the conductive substance can be a fluid, gel, metal, or organic material, where the fluid can be an environmental water sample, for example.

Embodiment 104: A sensor for determining a parameter of a fluid, comprising: a substrate having a top surface and a bottom surface; a working electrode comprising a chemically selective membrane, an ion-selective membrane, or a metal oxide, disposed on the bottom surface of the substrate; a reference electrode comprising the electrode of one or more of Embodiments 1 to 27, or 82, or 83, disposed on the bottom surface of the substrate, wherein the working electrode and reference electrode are electrically coupled when in contact with a fluid, where the fluid can be an environmental water sample, for example.

Embodiment 105: A solid state electrode comprising: an electrode having a surface; an interlayer coated on the surface, a selective membrane coated on the interlayer; wherein when the solid state working electrode is in electrical connection with a reference electrode and a conductive substance, wherein the electrode can detect a change in an analyte.

Embodiment 106: A solid state electrode comprising: an electrode having a surface; an interlayer coated on the surface, a selective membrane coated on the interlayer; wherein when the solid state working electrode is in electrical connection with a reference electrode and a conductive substance, wherein the electrode can detect a change in an analyte.

Embodiment 107: The solid state electrode of Embodiment 106, wherein the solid state electrode is a working electrode.

Embodiment 108: The solid state electrode of Embodiment 106, wherein a biorecognition element or redox process is used to generate analyte selectivity for the electrode.

Embodiment 109: A solid state electrode comprising: an electrode having a surface; a selective membrane coated on the surface; a biorecognition element or redox process is used to generate analyte selectivity; wherein when the solid state working electrode is in electrical connection with a reference electrode and a conductive substance, wherein the electrode can detect a change in an analyte.

Embodiment 110: The sensor of any one or more of Embodiments 1-27, 82, 83, 103, 104, 105, 106, or 109, wherein multiple sensors share a reference electrode.

Embodiment 111: The sensor of any one or more of Embodiments 1-27, 82, 83, 103, 104, 105, 106, or 109, wherein the metal is mercury, the compound of a metal is mercury chloride, and the nanoparticles are zinc oxide.

Embodiment 112: The sensor of any one or more of Embodiments 1-27, 82, 83, 103, 104, 105, 106, or 109, wherein the metal is mercury, the compound of a metal is mercury chloride, and the nanoparticles are silicon oxide.

Embodiment 113: The sensor of any one or more of Embodiments 1-27, 82, 83, 103, 104, 105, 106, or 109, wherein the metal is mercury, the compound of a metal is mercury chloride, and the nanoparticles are inorganic quantum dots.

Embodiment 114: The sensor of any one or more of Embodiments 1-27, 82, 83, 103, 104, 105, 106, or 109, wherein the metal is mercury, the compound of a metal is mercury chloride, and the nanoparticles are carbon quantum dots.

Embodiment 115: The sensor of any one or more of Embodiments 1-27, 82, 83, 103, 104, 105, 106, or 109, wherein the metal is mercury, the compound of a metal is mercury chloride, and the nanoparticles are fullerenes.

Embodiment 116: The sensor of any one or more of Embodiments 1-27, 82, 83, 103, 104, 105, 106, or 109, wherein the metal is mercury, the compound of a metal is mercury chloride, and the nanoparticles are carbon nanotubes.

Embodiment 117: The sensor of any one or more of Embodiments 1-27, 82, 83, 103, 104, 105, 106, or 109,: wherein the metal is mercury, the compound of a metal is mercury chloride, and the nanoparticles are graphene oxide.

Embodiment 118: The sensor of any one or more of Embodiments 1-27, 82, 83, 103, 104, 105, 106, or 109, wherein the metal is mercury, the compound of a metal is mercury chloride, and the nanoparticles are vanadium oxide nanoparticles.

Embodiment 119: The sensor of any one or more of Embodiments 1-27, 82, 83, 103, 104, 105, 106, or 109, wherein the metal is mercury, the compound of a metal is mercury chloride, and the nanoparticles are cerium oxide nanoparticles.

Embodiment 120: The sensor of any one or more of Embodiments 1-27, 82, 83, 103, 104, 105, 106, or 109, wherein the metal is mercury, the compound of a metal is mercury chloride, and the nanoparticles are europium oxide nanoparticles.

Embodiment 121: The sensor of any one or more of Embodiments 1-27, 82, 83, 103, 104, 105, 106, or 109, wherein the metal is mercury, the compound of a metal is mercury chloride, and the nanoparticles are diamond nanoparticles.

Embodiment 122: The sensor of any one or more of Embodiments 1-27, 82, 83, 103, 104, 105, 106, or 109, wherein the metal is silver, the compound of a metal is silver chloride, and the nanoparticles are zinc oxide.

Embodiment 123: The sensor of any one or more of Embodiments 1-27, 82, 83, 103, 104, 105, 106, or 109, wherein the metal is silver, the compound of a metal is silver chloride, and the nanoparticles are silicon oxide.

Embodiment 124: The sensor of any one or more of Embodiments 1-27, 82, 83, 103, 104, 105, 106, or 109, wherein the metal is silver, the compound of a metal is silver chloride, and the nanoparticles are inorganic quantum dots.

Embodiment 125: The sensor of any one or more of Embodiments 1-27, 82, 83, 103, 104, 105, 106, or 109, wherein the metal is silver, the compound of a metal is silver chloride, and the nanoparticles are carbon quantum dots.

Embodiment 126: The sensor of any one or more of Embodiments 1-27, 82, 83, 103, 104, 105, 106, or 109, wherein the metal is silver, the compound of a metal is silver chloride, and the nanoparticles are fullerenes.

Embodiment 127: The sensor of any one or more of Embodiments 1-27, 82, 83, 103, 104, 105, 106, or 109, wherein the metal is silver, the compound of a metal is silver chloride, and the nanoparticles are carbon nanotubes.

Embodiment 128: The sensor of any one or more of Embodiments 1-27, 82, 83, 103, 104, 105, 106, or 109, wherein the metal is silver, the compound of a metal is silver chloride, and the nanoparticles are graphene oxide.

Embodiment 129: The sensor of any one or more of Embodiments 1-27, 82, 83, 103, 104, 105, 106, or 109, wherein the metal is silver, the compound of a metal is silver chloride, and the nanoparticles are vanadium oxide nanoparticles.

Embodiment 130: The sensor of any one or more of Embodiments 1-27, 82, 83, 103, 104, 105, 106, or 109, wherein the metal is silver, the compound of a metal is silver chloride, and the nanoparticles are cerium oxide nanoparticles.

Embodiment 131: The sensor of any one or more of Embodiments 1-27, 82, 83, 103, 104, 105, 106, or 109, wherein the metal is silver, the compound of a metal is silver chloride, and the nanoparticles are europium oxide nanoparticles.

Embodiment 132: The sensor of any one or more of Embodiments 1-27, 82, 83, 103, 104, 105, 106, or 109, wherein the metal is silver, the compound of a metal is silver chloride, and the nanoparticles are diamond nanoparticles.

Embodiment 133: The sensor of any one or more of Embodiments 1-27, 82, 83, 103, 104, 105, 106, or 109, wherein the metal is silver, the compound of a metal is silver chloride, and the nanoparticles are gold nanoclusters.

Embodiment 134: The sensor of any one or more of Embodiments 1-27, 82, 83, 103, 104, 105, 106, or 109, wherein the metal is silver, the compound of a metal is silver chloride, and the nanoparticles are silver nanoclusters.

Embodiment 135: The sensor of any one or more of Embodiments 1-27, 82, 83, 103, 104, 105, 106, or 109, wherein the metal is silver, the compound of a metal is silver chloride, and the nanoparticles are gold nanoparticles.

Embodiment 136: The sensor of any one or more of Embodiments 1-27, 82, 83, 103, 104, 105, 106, or 109, wherein the metal is silver, the compound of a metal is silver chloride, and the nanoparticles are silver nanoparticles.

Embodiment 137: The sensor of any one or more of Embodiments 1-27, 82, 83, 103, 104, 105, 106, or 109, wherein the metal is silver, the compound of a metal is silver chloride, and the nanoparticles are titanium dioxide nanoparticles.

Embodiment 138: The sensor of any one or more of Embodiments 1-27, 82, 83, 103, 104, 105, 106, or 109, wherein the metal is copper, the compound of a metal is copper sulfate, and the nanoparticles are zinc oxide.

Embodiment 139: The sensor of any one or more of Embodiments 1-27, 82, 83, 103, 104, 105, 106, or 109, wherein the metal is copper, the compound of a metal is copper sulfate, and the nanoparticles are silicon oxide.

Embodiment 140: The sensor of any one or more of Embodiments 1-27, 82, 83, 103, 104, 105, 106, or 109, wherein the metal is copper, the compound of a metal is copper sulfate, and the nanoparticles are inorganic quantum dots.

Embodiment 141: The sensor of any one or more of Embodiments 1-27, 82, 83, 103, 104, 105, 106, or 109, wherein the metal is copper, the compound of a metal is copper sulfate, and the nanoparticles are carbon quantum dots.

Embodiment 142: The sensor of any one or more of Embodiments 1-27, 82, 83, 103, 104, 105, 106, or 109, wherein the metal is copper, the compound of a metal is copper sulfate, and the nanoparticles are fullerenes.

Embodiment 143: The sensor of any one or more of Embodiments 1-27, 82, 83, 103, 104, 105, 106, or 109, wherein the metal is copper, the compound of a metal is copper sulfate, and the nanoparticles are carbon nanotubes.

Embodiment 144: The sensor of any one or more of Embodiments 1-27, 82, 83, 103, 104, 105, 106, or 109, wherein the metal is copper, the compound of a metal is copper sulfate, and the nanoparticles are graphene oxide.

Embodiment 145: The sensor of any one or more of Embodiments 1-27, 82, 83, 103, 104, 105, 106, or 109, wherein the metal is copper, the compound of a metal is copper sulfate, and the nanoparticles are vanadium oxide nanoparticles.

Embodiment 146: The sensor of any one or more of Embodiments 1-27, 82, 83, 103, 104, 105, 106, or 109, wherein the metal is copper, the compound of a metal is copper sulfate, and the nanoparticles are cerium oxide nanoparticles.

Embodiment 147: The sensor of any one or more of Embodiments 1-27, 82, 83, 103, 104, 105, 106, or 109, wherein the metal is copper, the compound of a metal is copper sulfate, and the nanoparticles are europium oxide nanoparticles.

Embodiment 148: The sensor of any one or more of Embodiments 1-27, 82, 83, 103, 104, 105, 106, or 109, wherein the metal is copper, the compound of a metal is copper sulfate, and the nanoparticles are diamond nanoparticles.

Embodiment 149: The sensor of any one or more of Embodiments 1-27, 82, 83, 103, 104, 105, 106, or 109, wherein the metal is copper, the compound of a metal is copper sulfate, and the nanoparticles are gold nanoclusters.

Embodiment 150: The sensor of any one or more of Embodiments 1-27, 82, 83, 103, 104, 105, 106, or 109, wherein the metal is copper, the compound of a metal is copper sulfate, and the nanoparticles are silver nanoclusters.

Embodiment 151: The sensor of any one or more of Embodiments 1-27, 82, 83, 103, 104, 105, 106, or 109, wherein the metal is copper, the compound of a metal is copper sulfate, and the nanoparticles are gold nanoparticles.

Embodiment 152: The sensor of any one or more of Embodiments 1-27, 82, 83, 103, 104, 105, 106, or 109, wherein the metal is copper, the compound of a metal is copper sulfate, and the nanoparticles are silver nanoparticles.

Embodiment 153: The sensor of any one or more of Embodiments 1-27, 82, 83, 103, 104, 105, 106, or 109, wherein the metal is copper, the compound of a metal is copper sulfate, and the nanoparticles are titanium dioxide nanoparticles.

Embodiment 154: The sensor of any one or more of Embodiments 1-27, 82, 83, 103, 104, 105, 106, or 109, wherein the metal is copper, the compound of a metal is copper sulfate, and the nanoparticles are titanium dioxide nanoparticles.

Embodiment 155: The sensor of any one or more of Embodiments 1-27, 82, 83, 103, 104, 105, 106, or 109, wherein the strongly binding polymer is PVB (polyvinyl butyral).

Embodiment 156: The sensor of any one or more of Embodiments 1-27, 82, 83, 103, 104, 105, 106, or 109, wherein the strongly binding protein is an adhesive proteins, a mussel protein, a fibrinogen, a protofilament, amyloid fibrils, amyloid nanofibrils, or a combination comprising at least one of the foregoing.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention.

All ranges disclosed herein are inclusive of the endpoints, and the endpoints are independently combinable with each other. “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or” unless clearly stated otherwise. It is to be understood that the described elements may be combined in any suitable manner in the various embodiments.

The term amine includes -NR1 R2 groups wherein R1 and R2 are each independently selected from hydrogen and alkyl groups having from one to four carbon atoms; and one or more thiol groups (—SH). The term “alkyl” includes branched or straight chain, unsaturated aliphatic C1-4 hydrocarbon groups e.g., methyl, ethyl, n-propyl, i-propyl.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A solid state electrode comprising:
a metal electrode having a surface;
a nanocomposite coated on at least a portion of the surface, the nanocomposite comprising:
a compound of the metal used in the electrode, and nanoparticles, a protein, a polymer, or a combination comprising at least one of the foregoing;
wherein when the solid state electrode is in electrical connection with a working electrode and a conductive substance, the solid state electrode can detect a change in chemical composition of the conductive substance, and the potential of the solid state electrode is stable to within 5 millivolts, preferably within 3 millivolts over a period of 20 minutes.

2. The solid state electrode of claim 1, wherein the conductive substance is a fluid, metal, material, or gel.

3. The solid state electrode of claim 1, further comprising a fluid in fluid communication with the metal electrode, and wherein the fluid is a body fluid.

4. The solid state electrode of claim 1, wherein the solid state electrode can detect a change in pH of less than or equal to 0.5 pH units, and in one embodiment less than or equal to 0.1 pH units.

5. The solid state electrode of claim 1, wherein the nanoparticles are carbon nanoparticles or metal nanoparticles.

6. The solid state electrode of claim 1, wherein the nanoparticles are modified, wherein a hydrophobic compound comprising an amine group and a thiol group is covalently bonded to the nanoparticles.

7. The solid state electrode of claim 6, wherein the hydrophobic compound is 4-aminothiophenol or 5-amino-2-mercaptobenzimidazole.

8. The solid state electrode of claim 1, wherein the nanoparticles have an average diameter of less than or equal to 20 nanometers.

9. A method of making a solid state electrode, comprising:
providing a metal electrode having a surface;
attaching a nanocomposite comprising a compound of the metal used in the electrode, and
nanoparticles, a protein, a polymer, or a combination comprising at least one of the foregoing, onto at least a portion of the electrode surface.

10. The method of claim 9, wherein attaching is physical deposition or electrochemical deposition.

11. The method of claim 9, further comprising:
applying a voltage to the surface in an acid solution, forming a coated surface comprising a compound of the metal used in the electrode; and
electrochemically depositing the nanocomposite onto the coated surface comprising the compound of the metal used in the electrode.

12. The method of claim 9, wherein the nanocomposite comprises carbon nanoparticles.

13. The method of claim 9, wherein the nanocomposite comprises metal nanoparticles.

14. The method of claim 9, wherein the nanocomposite comprises copper sulfate and nanoparticles.

15. The method of claim 9, wherein the nanocomposite comprises mercury chloride and nanoparticles.

16. The method of claim 9, wherein the nanocomposite comprises silver chloride and nanoparticles.

17. The method of claim 9, wherein attaching comprises:
contacting an aqueous solution of potassium chloride and nanoparticles with the surface;
applying a voltage to the surface, forming a compound of the metal used in the metal electrode-nanoparticle composite coated surface.

18. The method of claim 9, wherein the nanocomposite comprises nanoparticles, and wherein attaching comprises:
mixing the nanoparticles with an oxidizing agent in a solution, to form a solution of oxidizing agent and nanoparticles; and
applying the solution of oxidizing agent and nanoparticles to the surface to deposit the compound of the metal used in the metal electrode-nanoparticle composites.

19. The method of claim 9, wherein the nanocomposite comprises an adhesive protein and nanoparticles, and the method further comprises mixing the adhesive protein and nanoparticles with an oxidizing agent prior to attaching the nanocomposite onto the surface.

20. The method of claim 9, wherein the nanoparticles are modified nanoparticles, wherein a hydrophobic compound comprising an amine group and a thiol group is covalently bonded to the nanoparticles.

21. A biosensor for determining a parameter of a conductive substance, comprising:
a substrate having a top surface and a bottom surface;
a working electrode comprising a membrane, a selective membrane, an ion-selective membrane or a metal oxide, disposed on the top surface of the substrate;
a reference electrode comprising the solid state electrode of claim 1, disposed on the surface of the substrate, wherein the working electrode and reference electrode are electrically coupled when in contact with a conductive substance.

22. The biosensor of claim 21, wherein an interlayer is coated on the surface of the working electrode below the selective membrane, comprising of a hydrophobic conducting polymer; a hydrophobic metal oxide layer; nanoparticles; or nanoparticles modified with hydrophobic ligands; or a combination comprising one or more of the foregoing.

23. The biosensor of claim 22, wherein the interlayer comprises a conducting polymer.

24. The biosensor of claim 23, wherein the conducting polymer comprises poly(3,4-diethylenedioxythiophene) (PEDOT), polypyrrole, polyaniline, polythiophene, polyoctylthiophene (POT), P3HT, polytetrafluoroethylene, or a combination comprising at least one of the foregoing.

25. The biosensor of claim 22, wherein a hydrophobic ligand comprises Thiophenol, 2-Napthalenethiol, 9-Anthracenethiol, or a combination comprising at least one of the foregoing.

26. The biosensor of claim 21, wherein a biorecognition element is coated on the membrane of the working electrode.

27. The biosensor of claim 21, wherein the membrane is mixed with a polymer that has been imprinted with an analyte.

28. The biosensor of claim 21, wherein the conductive substance is a body fluid, and wherein the biosensor can be attached to the skin of a human or mammal.

29. The biosensor of claim 28, wherein the parameter of a body fluid is the level of H+, Na+, Mg2+, NO3−, K+, NH4+, Ca2+, Cl−, carbonate, bicarbonate, proteins, lipids, DNA, RNA, hormones, estrogen, progesterone, testosterone, androstenedione, beta-human chorionic gonadotrophin (hCG), cortisol, creatinine, urea, glucose, lactic acid, acids, salts, cations, cytokines, dopa, dopamine, drugs, opiates, buprenorphine, amphetamines, gamma hydroxybutyrates, ethanol, cocaine, alcohols, metabolites, xenometabolites, dioxins, xenobiotics, organic compounds, mycotoxins, metals, zinc, lead, mercury, cadmium, pthalates, arsenic, cyanide, BPA, environmental toxins, industrial metals, toxins, or a combination comprising at least one of the foregoing.

30. The biosensor of claim 21, wherein the conductive substance is sweat.

31. The biosensor of claim 21, wherein the biosensor is between 0.5 and 10 millimeters thick.

32. The biosensor of claim 21, further comprising a flushing system to reduce the concentration of ions on one or more electrodes.

33. The biosensor of claim 21, wherein the reference electrode is deposited on the substrate by screen printing, roll-to-roll printing, aerosol deposition, inkjet printing, thin film deposition, or electroplating.

34. The biosensor of claim 1, wherein the polymer is a strongly binding polymer, preferably PVB (polyvinyl butyral).

35. The biosensor of claim 1, wherein the protein is a strongly binding protein, preferably an adhesive protein, a mussel protein, a fibrinogen, a protofilament, amyloid fibrils, amyloid nanofibrils, or a combination comprising at least one of the foregoing.

36. The biosensor of claim 21, wherein the working electrode and reference electrode are each independently a noble metal, preferably silver, gold, platinum, palladium, copper, or carbon, or a combination comprising at least one of the foregoing.

37. The biosensor of claim 21, wherein the compound of a metal used in the reference electrode is mercury chloride, silver chloride, silver iodide, copper sulfate, mercurous sulfate, or a combination comprising at least one of the foregoing.
